(19) United States
(12) Patent Application Publication
(10) Pub. No.: US 2014/0161918 A1
(43) Pub. Date: Jun. 12, 2014

(54) Use of Adipose Septa Protein Modulators and Compositions Thereof

(71) Applicant: AVON PRODUCTS, INC., Suffern, NY (US)

(72) Inventors: Cheng S. Hwang, New Milford, NJ (US); Jola Idkowiak Baldys, Franklin Lakes, NJ (US); Uma Santhanam, Tenafly, NJ (US); John W. Lyga, Basking Ridge, NJ (US)

(73) Assignee: AVON PRODUCTS, INC., Suffern, NY (US)

(21) Appl. No.: 13/710,536

(22) Filed: Dec. 11, 2012

(51) Int. Cl. A61K 8/97; A61K 8/35; A61Q 19/08; A61Q 19/00; A61Q 19/06; A61K 8/31; A61K 8/49

(52) U.S. Cl. CPC A61K 8/97; A61K 8/31; A61K 8/35; A61K 8/4973; A61Q 19/00; A61Q 19/06; A61Q 19/08
USPC 424/764; 514/469; 424/725

(57) ABSTRACT

Methods of using adipose septa protein modulators to impart anti-aging benefits to the skin and/or improve skin conditions resulting from weakened or compromised adipose septa.

### CROSS-REFERENCE TO RELATED APPLICATIONS

This application is a continuation-in-part of, and claims priority to, U.S. patent application Ser. No. 61/360,083, filed on Jun. 30, 2010; Ser. No. 13/158,947, filed on Jun. 13, 2011; 61/428,272, filed on Dec. 30, 2010; Ser. No. 13/305,779, filed on Nov. 29, 2011; Ser. No. 13/216,626, filed on Aug. 24, 2011; and Ser. No. 12/827,001, filed on Jun. 30, 2010; the entirety of each which are incorporated by reference in their entirety for all purposes.

Additionally, this application is filed concurrently with and claims priority to PCT application Ser. No. ______, entitled “Callistephus chinensis extracts and methods of use for improving the condition and appearance of skin and other keratinous materials”, filed on Dec. 11, 2012 and naming Qian Zheng as first inventor; PCT application Ser. No. ______, entitled “Serissa japonica extracts and methods of use”, filed on Dec. 11, 2012 and naming Qian Zheng as first inventor; PCT application Ser. No. ______, entitled “Use of Melicope extracts to improve conditions caused by excess lipids”, filed on Dec. 11, 2012 and naming Cheng Hwang as first inventor; PCT application Ser. No. ______, entitled “Hoya carnosa extracts and methods of use”, filed on Dec. 11, 2012 and naming Qian Zheng as first inventor; PCT application Ser. No. ______, entitled “Stephanotis jasminoides extracts and methods of use”, filed on Dec. 11, 2012 and naming Permanan Raaj Khusial as first inventor; PCT application Ser. No. ______, entitled “Medemia nobilis extracts and methods of use”, filed on Dec. 11, 2012 and naming Qian Zheng as first inventor; and PCT application Ser. No. ______, entitled “Maesa japonica extracts and methods of use, filed on Dec. 11, 2012 and naming Siming Chen as first inventor; the entirety of each of which is incorporated by reference herein in its entirety for all purposes.

Finally, this application incorporates by reference in their entirety for all purposes the following U.S. patent application Ser No. 12/648,581, filed Dec. 29, 2009; Ser. No. 12/966,098, filed on Dec. 29, 2009; Ser. No. 12/345,707, filed on Dec. 30, 2008; and Ser. No. 12/158,188, filed on Feb. 28, 2006.

### FIELD OF INVENTION

The present invention relates generally to compositions for topical application to the skin which comprise at least one adipose septa protein modulator and the use of such compositions to provide benefits to the skin, in particular, improving the condition and appearance of skin affected by cellulite.

### BACKGROUND OF THE INVENTION

There is active interest in the cosmetics industry in developing products that may be applied topically to the skin to counteract adverse changes in the skin surface contour, i.e., cellulite or sagging of facial or neck skin such as periorbital bulging, bags surrounding the eyes. Cosmetic products that reverse or forestall such changes are increasingly in demand.

The skin, epidermis and dermis are supported by a layer of subcutaneous fat that insulates and cushions the body from external forces. The subcutaneous fat is comprised of adipocytes, fat cells, arranged in chambers or lobules separated by fibrous septa or adipose septa. In women, these fibrous septa run parallel to one another and are perpendicular to the skin generating columns of adipocytes. In men, the fibrous septa are in a criss-cross pattern. Over time the fibrous septa weakens. As lobules expand due to excessive fat accumulation in the adipocytes, they stretch the septa and surrounding connective tissues. Eventually, the septa lose elasticity and become inflexible and thereby constrain the movement of the attached skin. This results in areas where the skin is held fast and elsewhere the skin bulges outward, resulting in cellulite—the lumpy “orange peel” or “cottage cheese” appearance on the skin's surface. Women, because of the columnar orientation of their septa, are more susceptible to cellulite which tends to manifest on their buttocks, abdomen, thighs, and limbs. A number of factors can cause cellulite including e.g., hereditary, intestinal, circulatory, lymphatic, hormonal, and lifestyle factors. However, contrary to popular thought, cellulite is not a result of obesity, although obesity can exacerbate the irregular appearance of cellulite.

Further, the face and neck have several subcutaneous fat pads, such as the orbital, buccal, malar (cheek), jowl, forehead, nasolabial fold, and parapharyngial fat pads, that provide cushioning and fullness to the face and neck. Each of these fat pads is retained in place by adipose septa. The septa may weaken due to environmental, heredity, nutritional, or chronological factors leading to a downward shift of the fat pad due to gravity that leads to undesirable features such as pronounced jowls, bags or sagging, double chins, etc.

A specific example of this sagging is periorbital bulging. Around the eyes, where the skin is up to five (5) times thinner than elsewhere in the body, fibrous septa, in the form of the orbital septa, are still present and serve to retain the normal fat that helps support and encircle the eye balls in the orbital cavity. Over time the tissues around the eye including the orbital septa weaken. Due to this weakness, the fat normally encircling the eyeballs in the orbital cavity migrates forward and downwards into the lower eyelids which causes periorbital, sub and supra orbital, bulging, i.e., bags. Some tissue fluids may also accumulate in the space below the eyes due to gravity giving the suborbital bulging, i.e. under-eye bags, a swollen appearance. The appearance of under-eye bags may be exacerbated by several factors including lack of sleep, stress, allergies, and time of day.

Scleroderma is a systemic disease that affects many organ systems. It is most obvious in the skin. However, the gastrointestinal tract, the respiratory, renal, cardiovascular, and genitourinary systems, as well as numerous vascular structures, are involved frequently. The symptoms result from progressive tissue fibrosis and occlusion of the microvasculature by excessive production and deposition of types I and III collagens. As the disease progresses, the skin becomes taut, shiny, and hyper-pigmented; the face becomes mask-like; and telangiectases appear on the fingers, chest, face, lips, and tongue. Subcutaneous calcifications may develop on the fingertips and over bony eminences. The Merck Manual, 17th ed., p. The condition includes a group of connective tissue and rheumatic disorders, including localized scleroderma (morphea and linear scleroderma) and systemic scleroderma (limited scleroderma, diffuse scleroderma, and sine scleroderma).

Over the years, a variety of approaches for treating these skin irregularities have been offered. Numerous dermatologic creams, lotions, vitamins, and herbal supplements have been proposed. Further, private spas and salons have offered massages, scrubs, wraps, compresses, essential oils, and herbal products to address the irregular skin contours. Most of these therapies do not provide a lasting remedy to these skin irregularities and require multiple treatments on an ongoing basis, often at considerable expense, to maintain any effect. Surgical or mechanical remedies to these skin irregularities have also been proposed including liposuction, in the case of cellulite; blepharoplasty, in the case of under-eye bags; and facelifts (rhytidectomy), in the case of sagging facial or neck skin to provide longer lasting relief. In particular, a series of treatments related to breaking up sclerotic fibrous septa including mechanical massages, which applies pressure and suction to reposition adipose tissue, and subscision, which uses a large gauge needle to break-up fibrous septa in the subdermal region, have been proposed to treat cellulite. However, these treatments often pose the risk of serious side effects.

A need remains for cosmetic compositions which reduce skin irregularities associated with weakened adipose septa such as cellulite and sagging skin of the face or necks, such as periorbital bulging. It is therefore an object of the invention to provide new compositions and methods for restoring strength and elasticity to fibrous septa present within adipose tissues. It is a further object of the invention to improve the overall appearance of skin, including treating, reversing, and/or preventing skin irregularities, such as cellulite and sagging skin of the face or necks, such as periorbital bulging, by modulating heretofore unidentified adipose septa proteins with new cosmetic compositions.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF THE INVENTION

The current invention relates to a method for improving the appearance of skin affected by a skin irregularity resulting from adipose septa by topically applying thereto an effective amount of at least one modulator of an adipose septa protein, in a cosmetically acceptable vehicle for a time sufficient to achieve an improvement in the appearance of said skin. In one embodiment, the modulator upregulates an adipose septa protein, and in a further embodiment the adipose septa protein is biglycan, mimecan, prolargin, and/or Dermatopontin. In another embodiment the modulator downregulates an adipose septa protein, and in another embodiment the adipose septa protein is biglycan, prolargin, and/or dermatopotin.

In a further embodiment of the method of the current invention, the skin irregularity is cellulite, sagging facial skin, sagging neck skin, or scleroderma. In another embodiment, the skin irregularity is cellulite, and in one embodiment this cellulite is located on a thigh, buttocks, abdomen, hip, and/or upper arm region. In a further embodiment related to cellulite, the achievement in the appearance of the skin obtained from the current method is selected from the group consisting of: (a) reduction in the appearance of cellulite lumpiness and/or unevenness; (b) reduction in pitting appearance of cellulite upon squeezing; (c) reduction in the extent of area affected by cellulite; (d) prevention or delay in the recurrence of cellulite; (e) improvement in collagen disposition; or (f) improvement in adipocyte/fat tissue disposition. In yet another embodiment, the method of the current invention is used to reduce the re-occurrence of cellulite in an area previously affected by cellulite.

In yet another embodiment of the current invention, the skin irregularity is sagging of the facial skin, and in one embodiment is periorbital bulging. The improvement in appearance of the skin is selected from the group consisting of: (a) improvement in adipocyte/fat tissue disposition; (b) reduction in sagging of the facial skin; or (c) reduction in the extent of the area affected by sagging.

In one embodiment of the current invention, the modulator may be palmitoyl lysyl aminovaleroyl lysine, an extract of Amorphophallus campanulantus, an extract of Mademia nobilis, an extract of Erythrina indica, an extract of Ixora chinensis, an extract of Operculina turpethum, an extract of Portulaca oleracea, an extract of Tiliacora triandra, an extract of Melicope hayesii, Mellicope elleryana, and/or hybrids or combinations thereof, or any combination thereof. In a further embodiment of the current invention, at least two modulators are applied. In another embodiment, themodulator is not an extract of Tiliacora triandra, Melicope hayesii, Melicope elleryana, and/or hybrids or combinations thereof in certain embodiments of the current invention.

The adipose septa protein modulated in certain embodiments of the current invention may include asporin, biglycan, decorin, dermatopontin, fibromodulin, fibronectin, galectin-1, laminin beta 2, lumican, MAGP-4, mimecan (osteoglycin), nidogen-1, nidogen-2, prolargin, or any combination thereof.

In a further embodiment of the current invention, the modulator may be used in combination with at least one other anti-lipid agent, and in a further embodiment the at least one other anti-lipid agent comprises a Carnitine Palmitoyl Transferase-1 (CPT-1) stimulator. The modulator is applied in combination with at least one anti-cellulite agent in a further embodiment of the current invention. The anti-cellulite agent may comprise a phophodiesterase inhibitor, an adenylate cyclase activator, a lipolysis stimulator, a beta-adrenergic receptor agonist, an alpha-2-adreneric receptor antagonist, perilla oil, carnitine, creatine, or any combination thereof. In particular, the at least one anti-cellulite agent may be from the group consisting of a xanthine analog, forskolin, a Coleus forskohlii extract, a Hawthorne extract, a cola extract, isoproterenol, yohimbine, Ginkgo biloba extract, perilla oil, or any combination thereof. In yet another embodiment, the anti-cellulite agent is caffeine. The method according to the current invention may also use the modulator in combination with at least one collagen and/or elastin stimulator. The method according to the current invention may further include embodiments in which the modulator is used in combination with a retinoid.

Another embodiment is directed to the current method wherein the composition is applied at least once daily for a period of time sufficient to improve the appearance of the skin. In accordance with the current invention the composition may be a leave on composition. In certain embodiments of the current invention, the effective amount of the modulator is about 0.001% to about 25% by weight, and in one embodiment about 0.001% to about 1% by weight. Further, an embodiment of the current invention is directed to the use of the method of the current invention daily for a period of four weeks.

A further aspect of the current invention is directed to a method for screening active agents useful for improving the aesthetic appearance of skin that involves assaying candidate substances for their ability to modify adipose septa protein expression. In a further embodiment of this screening method, the assaying step involves incubating preadipocytes or at least partially differentiated adipocytes with said candidate substance and subsequently measuring the levels of mRNA encoding adipose septa protein expression. The step of measuring may be carried out by quantitative polymerase chain reaction (qPCR) in other embodiments of the current invention. In certain embodiments, the adipose septa proteins used within the screening are mimecan, biglycan, prolargin, and/or dermatopontin.

The current embodiment is further directed to a method of treating the skin comprising topically applying to an area of the skin in need thereof an effective amount of an active agent that modulates adipose septa protein expression, wherein the ability of said active agent to modulate adipose septa protein expression has been determined by an assay which measures the level of mRNA encoding adipose septa protein in a cell that has been contacted with said active agent.

These and other aspects of the present invention will be better understood by reference to the following detailed description.

### DESCRIPTION OF THE DRAWINGS

FIG. 1 is an HPLC profile of an extract of Tiliacora triandra.

FIG. 2 is an HPLC profile of an extract of Medemia nobilis

FIG. 3 is an HPLC profile of an extract of Ixora chinensis

FIG. 4 is an HPLC profile of an extract of Operculina turpethum

FIG. 5 is an HPLC profile of an extract of Erythrina indicia

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Therefore, specific structural and functional details disclosed herein are not to be interpreted as limiting, but merely as a representative basis for teaching one skilled in the art to variously employ the present invention.

The current invention provides for a new and novel method of treating, preventing, and/or forestalling various skin irregularities through the administration of adipose septa protein modulators to skin in need thereof. In particular, the adipose septa protein modulators seek to treat, prevent, or forestall skin irregularities, such as cellulite and/or sagging skin of the face or neck, such as periorbital bulging, by upregulating one or more adipose septa proteins and thereby rejuvenating, repairing, and/or strengthening the adipose septa or fibrous septa. The skin irregularities may be due to a weakening the adipose septa due to environmental, nutritional, heredity, or age related issues. Additionally, the adipose skin protein modulators seek to treat, prevent, or forestall skin irregularities, such as scleroderma, by downregulating one or more adipose septa proteins.

One aspect of the present invention relates to compositions for topical application which comprises an effective amount of an adipose septa protein modulator to treat, reverse, ameliorate and/or prevent various conditions characterized by the weakening of septa enclosing adipose tissue. Such benefits include without limitation, the following:

(a) reduction in the appearance of cellulite lumpiness and/or unevenness;

(b) reduction in pitting appearance of cellulite upon squeezing;

(c) reduction in the extent of area affected by cellulite;

(d) prevention or delay in the recurrence of cellulite;

(e) improvement in collagen disposition;

(f) improvement in adipocyte/fat tissue disposition;

(g) reduction in sagging of the skin; and/or

(h) reduction in the extent of the area affected by sagging skin.

In practice, the compositions of the invention are applied to skin in need of treatment, i.e., skin which suffers from a deficiency or loss in any of the foregoing attributes or which would otherwise benefit from improvement in any of the foregoing skin attributes.

In certain embodiments the compositions and methods of the invention are directed to the prevention, treatment, and/or amelioration of cellulite. In this case, the compositions are applied to skin in need of treatment, by which is meant skin exhibiting cellulite normally or upon squeezing. The compositions are applied directly to the area of the skin exhibiting cellulite. The compositions and methods are suitable for treating cellulite on any surface of the skin, including without limitation, the buttocks, thighs, hips, or limbs. Additionally, the compositions of the current invention can be used to treat, ameliorate, and/or prevent/delay the appearance of sagging facial or neck skin, in particular periorbital bulging, and are applied to the sagging area in need of treatment.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, sec. 201(i).

All terms used herein are intended to have their ordinary meaning unless otherwise provided.

As used herein, the term “consisting essentially of” is intended to limit the invention to the specified materials or steps and those that do not materially affect the basic and novel characteristics of the claimed invention, as understood from a reading of this specification. All percentages are by weight based on the total weight of the composition, unless otherwise indicated.

By “cosmetically acceptable” it is meant that a particular component is generally regarded as safe and nontoxic at the levels employed.

The term “active amount” refers to the amount of adipose septa protein modulator, absent diluent, solvent, carrier, filler or any other ingredient. An “amount effective” or an “effective amount” to provide a particular anti-aging benefit to the skin refers to the “active amount” of extract required to provide a clinically measurable improvement in the particular manifestation of skin aging when applied for a time sufficient to provide a clinically measurable improvement in the particular manifestation of weakened adipose septa.

The phrase “individual in need thereof” refers to a human who could benefit from improved dermal appearance or health, including males or females.

As used herein, the terms “prevent,” “preventing,” etc. mean delaying the onset of, hindering the progress of, hindering the appearance of, protection against, inhibiting or eliminating the emergence of, or reducing the incidence of various cosmetic or dermatologic conditions, damages, effects or symptoms. Use of the term “prevention” is not meant to imply that all subjects in a subject population administered the cosmetic composition will always be unaffected by or fail to develop the cosmetic or dermatologic conditions, damage, effect or symptom, but rather that the subject population will exhibit a reduction in the cosmetic or dermatologic damages, effects, or symptoms. For example, many flu vaccines are not 100% effective in preventing the flu in those administered the vaccine.

The term “modulator” encompasses any substance, including, without limitation, organic molecules; biomolecules (e.g., peptides, proteins, antibodies, nucleic acid oligomers, etc.); and combinations of substances, such as botanical extracts. The modulators regulate the cellular levels of at least one adipose septa protein, by which is meant that the cellular levels of adipose septa protein are either increased or decreased by the active agent. The term “modulation” may refer to up-regulation, induction, stimulation, potentiation, and/or relief of inhibition, as well as inhibition, attenuation and/or down-regulation or suppression. The modulators may be, without limitation, activators or agonists, which are compounds that, for example, bind to, stimulate, increase, open, activate, facilitate, enhance activation, sensitize, or up-regulate expression levels of genes or adipose septa proteins or peptides. The modulators may also be, without limitation, inhibitors or antagonists, which are, for example, compounds that bind to, partially or totally block stimulation, decrease, prevent, delay activation, inactivate, desensitize, or downregulate expression levels of genes or adipose septa proteins or peptides. The mechanism by which the protein level is modulated is not important.

As used herein, the term “expression levels” refers to an amount of a gene and/or protein that is expressed in a cell. As used herein, a “gene” includes a polynucleotide containing at least one open reading frame that is capable of encoding a particular polypeptide. As used herein, the terms “polynucleotide” is synonymous with “oligonucleotide” and includes polymeric forms of nucleotides of any length, either deoxyribonucleotides or ribonucleotides, or analogs thereof, including, without limitation, mRNA, DNA, cDNA, primers, probes, and the like.

As used herein, the term “essential oil” refers to the volatile ethereal fraction obtained from a plant or plant part by a physical separation process such as distillation or chromatographic separation. The essential oils are typically terpenoids often comprising monoterpenes and have the odor and flavor of the plant from which they were extracted. In certain embodiments, the essential oils may include, but are not limited to, caryophylene (C₁₅H₂₄), bicyclogermacrene (C₁₅H₂₂), zierone (C₁₅H₂₂O), or evidone (C₁₀H₁₂O₂).

Surprisingly, to date, the proteins of the adipose septa/fibrous septa have not been reported in the literature. As noted in Example 1 below, proteomic analysis of the adipose septa/fibrous septa indicated, with high confidence, that the adipose septa incorporates the following proteins: asporin, biglycan, decorin, dermatopin, fibromodulin, fibronectin, galectin-1, laminin beta 2, lumican, MAGP-4, mimecan (osteoglycin), nidgen-1, nidgen-2, and prolargin (the “adipose septa proteins”).

A number of the proteins comprising the adipose septa, e.g., asporin, biglycan, decorin, fibromodulin, lumican, and mimecan (osteoglycin), are proteoglycans. Proteoglycans contain a core protein to which is attached one or more glycosaminoglycan (GAG) side-chains. Biglycan, decorin, fibromodulin, and mimecan all belong to the small leucine-rich proteoglycan (SLRP) family. These proteoglycans are bifunctional molecules in which a protein moiety tends to bind collagen fibrils and the highly charged hydrophilic glycosaminoglycan moiety regulates the intrafibrillar spacing. Decorin and fibromodulin are essential to collagen fibril formation. Asporin is an additional SLRP, however it lacks the glycosaminoglycan moiety and is largely involved in the cartilage matrix. Lumican is a keratin sulfate proteoglycan found in the collagenous matrix throughout the body. Lumican may regulate collagen fibril organization and circumferential growth, and epidermal growth and repair.

Dermatopontin, also known as TRAMP, is a widely expressed noncollagenous protein component of the extracellular matrix. It regulates the interaction of TGF-beta and decorin and is involved in collagen matrix organization.

Microfibril associated glycoprotein-4 (MFAP4) has binding affinities for collagen and carbohydrates and is thought to be involved in cell adhesion or intracellular interactions.

Galectin-1 is part of carbohydrate binding proteins with an affinity for B-galactoside and has been identified as a regulator of the immune response within the extracellular matrix.

Fibronectin is a glycoprotein that contributes to the survival of cells. Within the extracellular matrix, it has a role in binding many proteins and can serve as a template for collagen deposition.

A number of the adipose septa proteins identified are implicated in the formation and maintenance of basement membranes as well. Laminin Beta 2, is part of a family of extracellular matrix glycoproteins that are the major noncollagenous constituent of basement membranes. These proteins have been implicated in a wide variety of biological processes including cell adhesion, differentiation, migration, signaling, neurite outgrowth and metastasis. Nidogen, or entactin, 1 and 2 are cell adhesion mediating sulphated glycoproteins that are an integral component of the basement membrane and associate with laminin and collagen. Prolargin is a leucine-rich repeat protein present in connective tissue extracellular matrix. This protein functions as a molecule anchoring basement membranes to the underlying connective tissue.

Each of these proteins have a role in the overall quality of the adipose septa, and the modulation of at least one of these adipose septa proteins may repair, maintain, or enhance the integrity of the adipose septa thereby preventing, forestalling, or remedying skin irregularities, such as cellulite and sagging facial or neck skin, in particular periorbital bulging. In one embodiment, more than one adipose septa protein is modulated, in one embodiment at least two or more are modulated. Further, the modulators may upregulate or downregulate the adipose septa protein, and further one modulator may both upregulate certain adipose septa proteins while down regulating others. Also, where more than one adipose septa protein modulator is used within the current method, at least one modulator may upregulate a first adipose septa protein and at least one modulator may down regulate a second adipose septa protein. The above noted combinations of adipose septa protein modulators may be used to cumulative or synergistic effect when addressing skin irregularities due to the adipose septa. Additionally, in certain embodiments one or more of the adipose septa proteins are not modulated by an adipose septa protein modulator, in certain embodiments biglycan, decorin, fibromodulin, lumican, fibronectin, and/or laminin are not upregulated, and in further embodiments the proteoglycans are not upregulated. The above noted combinations of adipose septa protein modulators may be used to cumulative or synergistic effect when addressing skin irregularities due to the adipose septa.

The discovery of the main proteins comprising adipose septa has led to a screening method for identifying potential adipose septa protein modulators. In one embodiment, an assay is provided for determining the expression levels of an adipose septa protein after a cell has been treated, incubated, or otherwise contacted with a candidate substance. The term “candidate substance” refers to any substance that is tested for activity as a modulator of adipose septa proteins, whether or not the substance is suspected of possessing such activity. The cell can be any cell that expresses adipose septa proteins. In one embodiment, the cell is a mammalian preadipocyte. In another embodiment, the cell is a mammalian adipocyte. In further embodiments, the cell is a human or mouse cell. After the cell has been incubated with a candidate substance for a sufficient length of time to provide a measurable change in expression levels, which will typically be at least one hour, and more typically from about 72 hours to 144 hours (3 to 6 days) it is then lysed to release the cellular components, such as an adipose septa protein and mRNA encoding those proteins. The amount of an adipose septa protein or any subunit thereof may then be measured by any suitable technique for detection and quantitation of peptides and proteins and/or polynucleotides (e.g., mRNA).

In one embodiment, the methods for measuring expression levels of adipose septa proteins involve the quantitation of mRNA expression. Suitable methods for determining mRNA expression include quantitative PCR (QPCR), real-time QPCR, reverse transcription PCR (RT-PCR), and quantitative reverse transcription PCR (QRT-PCR), as are well-known in the art. As described in detail in U.S. Pat. Nos. 7,101,663 and 7,662,561, the disclosures of which are hereby incorporated by reference in their entirety, a quantitative reverse transcriptase polymerase chain reaction (QRT-PCR) for detecting mRNA may include the steps of: (a) incubating an RNA sample from the cellular lysate with a reverse transcriptase and a high concentration of a target sequence-specific reverse transcriptase primer under conditions suitable to generate cDNA; (b) subsequently adding suitable polymerase chain reaction (PCR) reagents to the reverse transcriptase reaction, including a high concentration of a PCR primer set specific to the cDNA and a thermostable DNA polymerase to the reverse transcriptase reaction; and (c) cycling the PCR reaction for a desired number of cycles and under suitable conditions to generate PCR products (“amplicons”) specific to the cDNA. The products of the QRT-PCR process may be compared after a fixed number of PCR cycles to determine the relative quantity of the RNA species as compared to a given reporter gene, for example, by Southern blotting. More typically, the progress of the PCR reaction is monitored by analyzing the relative rates of amplicon production for each PCR primer set, for example, by (1) non-specific fluorescent dyes that intercalate with any double-stranded DNA, and/or (2) sequence-specific DNA probes consisting of oligonucleotides that are labeled with a fluorescent reporter which permits detection only after hybridization of the probe with its complementary DNA target. The mRNA may be any mRNA known to one of ordinary skill of the art that is associated with the adipose septa protein of interest.

Additionally, Example 2 below, provides another method for determining the expression levels of an adipose septa protein after a cell has been treated, incubated, or otherwise contacted with a candidate substance.

The level of expression in the above disclosed methods of determining adipose septa protein expression levels may be compared to controls that are not treated with the candidate substance to determine the relative degree of modulation. In some embodiments, the candidate substance will up-regulate mRNA expression by at least about 10%, more suitably at least about 20%, at least about 30%, at least about 40%, or at least about 50%. In further embodiments, the candidate substance will up-regulate mRNA expression by at least about 60%, at least about 70%, at least about 80%, at least about 90%, or at least about 100%. Candidate substances meeting these criteria may be selected for use of for further evaluation.

Adipose septa modulators, identified in accordance with the procedures noted above as shown by Example 2 below, can include naturally occurring or synthetic peptides, amino acids, and chemicals entities such as, but not limited to, palmitoyl lysyl aminovaleroyl lysine. Further, the adipose septa modulators may include botanical extracts such as an extracts of: Amorphophallus campanulatus, Medemia nobilis, Erythrina indica, Ixora chinensis, Operculina turpethum, Portulaca oleracea, Tiliacora triandra, Melicope (hayesii, elleryana, or hybrids and/or combinations thereof), Butea frondosa or any combinations thereof.

Amorphophallus campanulatus (“OI Kach”) is a perennial herb widely distributed in Bangladesh, India, and Africa, with rounded tuberous root stocks (corm). The roots have traditionally been used to treat piles, abdominal pains, tumours, enlargement of the spleen, asthma, and rheumatism, and is described, for example, in U.S. patent application Ser. No. 13/158,947, filed on Jun. 30, 2010, the entirety of which is incorporated by reference herein for all purposes.

Medemia nobilis is a dioecious palm that is native to Madagascar. It produces inflorescences, about 1 m long, with thick, catkin-like branches covered with inconspicuous flowers, and is described, for example, in U.S. patent application Ser. Nos. 13/305,779 and 13/216,626, filed on Dec. 30, 2010 and Aug. 24, 2011, respectively, the entirety of which are incorporated by reference for all purposes.

Erythrina indica (Indian Coral Tree) is a middle-sized quick growing tree found in Bengal and many parts of India. The flowers of the tree are bourne in dense racemes, coral red and used traditionally for the treatment of liver trouble, joint pain, dysentery, convulsions and as diuretic, laxative, and anthelmic, and is described, for example, in U.S. patent application Ser. No. 12/648,581, filed Dec. 29, 2009; Ser. No. 12/966,098, filed on Dec. 29, 2009; and Ser. No. 12/827,001, filed on Jun. 30, 2010, the entirety of each of which is incorporated by reference for all purposes.

Ixora chinensis is a flowering plant native to southern China that is characterized by its almost stalkless leaves and red flowers. It is used to treat various ailments like rheumatism and wounds, and is described, for example, in U.S. patent application Ser. No. 13/158,947, filed on Jun. 30, 2010, the entirety of which is incorporated by reference for all purposes.

Operculina turpethum (“Trivit”) is a large stout perennial twinner that has miky juice and fleshy roots. It is thought to have activity against liver disorders, and also has anthelmintic, expectorant, antipyretic, anti-inflammatory, and purgative properties, and is described, for example, in U.S. patent application Ser. No. 13/158,947, filed on Jun. 30, 2010, the entirety of which is incorporated by reference for all purposes.

Portulaca oleracea (“Portulaca”) is an annual herbaceous plant which originates from the region extending from the western Himalayas to southern Russia and Greece. Portulaca is eaten as a salad and vegetable around the world and used medicinally for a variety of conditions that include headache, stomach ache, painful urination, enteritis, mastitis, lack of milk flow in nursing mothers and in postpartum bleeding. Externally it is used to treat burns, earache, insect stings, inflammations, skin sores, ulcers, pruritis, eczema and abscesses. Leung & Foster, 1996, and is described, for example, in U.S. patent application Ser. No. 12/827,001, filed on Jun. 30, 2010, the entirety of which is incorporated by reference for all purposes.

Tiliacora triandra (“Yanang”) is a species of flowering plant native to mainland Southeast Asia and used particularly in the cuisines of northeast Thailand and Laos. In traditional Southeast Asian medicine, Tiliacora triandra has been used as an herbal medicine for fever relief, alcohol intoxication, inflammation, and bacterial/fungal infection. For instance, the use of Tiliacora triandra Diels against plasmodium falciparum (cause malaria in humans) is disclosed in Pavanand et al., Phytother. Res., 3, 215-217 (1989), and is described, for example, in U.S. patent application Ser. No. 12/345,707, filed on Dec. 30, 2008; Ser. No. 13/158,947, filed on Jun. 30, 2010; and Ser. No. 12/827,001, filed on Jun. 30, 2010, the entirety of each of which is incorporated by reference for all purposes.

Melicope refers to a diverse genus of the family Rutaceae family that includes 233 species, ranging from small shrubs to large canopy trees found in Madagascar, mainland Asia (Indo-Himilayan region), Hawaii, throughout Malesia, Australia, parts of the western Pacific, south to New Zealand. The plants of the genus are notable for their conspicuously oil-gland-dotted leaves. See e.g. Brophy, J. Essent. Oil Res. (2004), vol. 16, pp 286-293, hereby incorporated by reference in its entirety. Hayesii plants are commonly referred to as “Corkwood” or “Doughwood” in Australia and as “Alani” in Hawaii). Melicope hayesii is a shrub in the Melicope genus and the Rutaceae family that may be found in the wild on the north coast of New South Wales (NSW), Australia. Melicope elleryana is a tree that can reach heights of up to 35 meters found along creeks, swamp forests, or vineforests through Malesia and Australia. Further, hybrids of the various species of Melicope may occur naturally through cross-pollenation or through active cultivation i.e., planned cross-pollenation and/or grafting, and exhibit varying degrees of attributes from the constituent species including for example essential oils. The leaves of Melicope have been reported to have an anti-inflammatory effect, and are described, for example, in U.S. Pat. No. 7,592,024, the entirety of which is incorporated by reference for all purposes. Brophy, J. Essent. Oil Res. (2004), vol. 16, pp 286-293, hereby incorporated by reference in its entirety, provides essential oil profiles for species of Melicope including Melicope hayesii and Melicope elleryana. In one embodiment, the Melicope extract (e.g. essential oil) may have an essential oil profile that includes carophylene, bicyclogermacrene; zierone, and/or evidone. In one embodiment, the essential oil profile may include about 4.0 to about 6.0% by weight of carophyllene, about 0.5 to about 1.5% by weight of bicyclogermacrene, about 25 to about 30% by weight of zierone, and/or about 10 to about 15% by weight of evodone; and in another embodiment the essential oil profile may include, about 5.0 to about 6.0% by weight of carophyllene, about 0.75 to about 1.25% by weight of bicyclogermacrene, about 26 to about 29.5% by weight of zierone, and/or about 11 to about 15% by weight of evodone. In a further embodiment, the essential oil profile of Melicope extract may include about 4.0 to about 6.0% by weight of carophyllene, about 0.5 to about 1.5% by weight of bicyclogermacrene, about 25 to about 30% by weight of zierone, and about 10 to about 15% by weight of evodone; and in another embodiment the essential oil profile may include, about 5.0 to about 6.0% by weight of carophyllene, about 0.75 to about 1.25% by weight of bicyclogermacrene, about 26 to about 29.5% by weight of zierone, and about 11 to about 15% by weight of evodone.

The above-noted extracts contain a number of active compounds, one or more of which upregulate one or more of the adipose septa proteins. In certain embodiments of the current invention, adipose septa protein modulators may exclude one or more of the above-noted compounds and/or extracts, and in another embodiment extracts of Tiliacora triandra and/or Melicope (hayesii, elleryana, and/or hybrids or combinations thereof) are excluded.

The plant materials may be in any form including, but not limited to, the whole plant, a dried plant, a ground plant, or parts thereof, including but not limited to, seeds, needles, leaves, roots, bark, cones, stems, rhizomes, callus cells, protoplasts, flowers, and meristems, or components and/or constituents found in, or isolated from, the natural plant material, and/or portions of the plant, or any combinations thereof. In one embodiment, the natural plant material is in the form of an extract derived from the whole plant or from a select portion of the plant, such as the leaves of the plant. It is to be understood that “natural plant material” also includes an ingredient, component, constituent, or extract derived from the natural plant material.

Specifically, the botanical component is derived from raw materials collected from the plants, which may contain the desired constituent(s), such as seeds, needles, leaves, roots, bark, cones, stems, rhizomes, callus cells, protoplasts, organs and organ systems, and meristems. In certain embodiments, the raw materials collected from the plants are ground to small particle sizes. In addition, the raw materials may be dried to reduce water content. The raw materials may be dried by a number of different means, such as, for example, air-dried, oven-dried, rotary evaporated under vacuum or lyophilized.

The extract of the above-noted plants may be obtained by distilling the raw materials with a stripping agent. The stripping agent may be a liquid that is miscible, immiscible, or partially miscible with the desired extract from the plants. Suitable stripping agents include, but are not limited to, water; alcohols (such as methanol, ethanol, propanol, butanol and the like); glycols; ethers (such as diethyl ether, dipropyl ether, and the like); esters (such as butyl acetate, ethyl acetate, and the like); ketones (such as acetone, ethyl methyl ketone, and the like); dimethyl sulfoxide; acetonitrile; other organic solvents; and combinations thereof. In one embodiment, the stripping agent is immiscible with the desired extract (e.g., essential oil) from the plant. In a further embodiment, the stripping agent is water. The extract is obtained by steam distillation in yet another embodiment. The extract (e.g., essential oil) may be collected by phase separation from the stripping agent. It is believed that the stripping agent increases the overall vapor pressure of a distillation system for obtaining an extract and thereby reducing the boiling point of the desired product, the extract.

In other embodiments, the botanical component may be in the form of an extract obtained by solvent extraction, in one embodiment obtained by an organic solvent extraction. Briefly, the organic solvent extraction method involves washing and extracting the raw materials, which may be whole or ground into small particle sizes, using an organic solvent. Non-limiting examples of organic solvents include methanol, ethanol, isopropanol, dichloromethane, chloroform, hexane, xylene, and petroleum ether. An extracting machine may be used for organic solvent extraction as is well known in the field. The raw materials are pushed in the extracting machine by a thruster, which slowly moves the plant raw materials forward. Organic solvent (e.g., ethanol) may be added into the machine through a solvent inlet at the top of a waste discharge outlet. Due to the difference in gravity and equilibrium, the solvent flows toward the raw material inlet, soaks the materials and flows out from the opposite side of the solvent inlet. Since the plant materials and the solvent move in opposite directions against each other, the plant materials are constantly immersed in a solution that contains a low-concentration of extract. As a result of equilibrium, high yield of plant constituent(s) may be achieved by continuously extracting the plant material against the low-concentration solution.

An extraction time suitable to extract the plant constituents is used, typically between about 1-10 hours is suitable, and in another embodiment is between about 2-8 hours, and in a further embodiment is between about 3-6 hours. The temperature of extraction is between about 30° C.-100° C., in a further embodiment is between about 40° C.-70° C., and in yet another embodiment is between about 50° C.-60° C. The collected extract is then fine-filtered to remove debris, and may be used directly, or is concentrated, for example by distilling the solvent or by other conventional processing. The solution of extract actives may be rotary evaporated under vacuum or lyophilized. A typical extract's actives content is above about 25%, in a further embodiment above 50%, and the extract can also be provided as an essential oil or a concentrate having a semi-solid or solid consistency.

Similarly, aqueous-organic solvent extraction involves initially collecting raw materials from the above-noted plants, which may be whole or ground into small particle sizes. The ground plant material is soaked in aqueous solution that is acidic or alkaline, depending on the solubility and stability of the desired extract under acidic or alkaline (basic) conditions. For extraction under acidic conditions, an acid such as hydrochloric acid or sulfuric acid is added to water, e.g., at a concentration of about 3% (w/v). For extraction under alkaline conditions, an alkali such as sodium hydroxide or sodium carbonate is added to water. The extraction time and temperature of extraction are typically similar to that used in the organic solvent extraction method described above.

The extract is then collected and fine-filtered to remove debris. Alkaline agents (e.g., ammonia) or acidifying agents (e.g., sulfuric acid) may be added to the extract to neutralize the solution by adjusting the pH, depending on the acidity or alkalinity of the collected extract. The aqueous extract may be used directly, concentrated or dried. Alternatively, organic solvent may then be added to the neutralized solution to transfer the extract from an aqueous phase to an organic phase. Examples of such organic solvents include, but are not limited to, ethanol, isopropanol, butanol, pentanol, hexanol and xylene. The extract comprising the transferred extract actives dissolved in organic solvent may be used directly as an essential oil or a concentrate, or dried by a number of different means, such as, for example, air-dried, oven-dried, rotary evaporated under vacuum or lyophilized to a semi-solid or solid consistency.

It should also be noted that different plants containing different constituents can be mixed and extracted together. This process of mixed extraction can in one embodiment be used for extracting those plants containing constituents with similar solubility in the solvent used for extraction, such as ethanol. The mixture of extracts can be concentrated and stored in an appropriate solvent.

Examples of preparations of extracts are provided below in Example 3.

In another embodiment, extract as used herein, also includes “synthetic” extracts, i.e., various combinations of known plant components and/or constituents that are combined to substantially mimic the composition and/or activity of any one or more of the above-noted plant extracts of natural origin having adipose septa protein modulating activities. In one embodiment, the synthetic extracts have substantially the same number of active components as the natural plant material. The correspondence of the numerical incidence of actives between the synthetic extracts and the natural plant material may also be described in terms of “percent commonality.” The synthetic extract has about 50 percent or more commonality to the chemical composition of a plant or natural extract. In other words, the synthetic extract has about 50 percent or more of the active ingredients found in the plant or a natural extract. In further embodiments, the chemical composition of the synthetic extract has about 70 percent or more commonality to the chemical composition of a plant or a natural extract. Optimally, a synthetic extract has about 90 percent or more commonality to the chemical composition of a plant or a natural extract.

The cosmetic compositions according to the invention can be formulated in a variety of forms for topical application and will comprise from about 0.00001% to about 90% by weight of one or more actives that modulate adipose septa proteins, and in other embodiments will comprise such actives in an amount from about 0.001% to about 25% by weight, and in a further embodiment it will comprise such actives in an amount from about 0.001% to about 1% by weight.

Another embodiment of the invention encompasses compositions comprising a cosmetically or dermatologically acceptable formulation which is suitable for contact with living animal tissue, including human tissue, with virtually no adverse physiological effect to the user. Compositions embraced by this invention can be provided in any cosmetically and/or dermatologically suitable form, in one embodiment as a lotion or cream, but also in an anhydrous or aqueous base, as well as in a sprayable liquid form. Other suitable cosmetic product forms for the compositions of this invention include, for example, an emulsion, a cream, a balm, a gloss, a lotion, a mask, a serum, a toner, an ointment, a mousse, a patch, a pomade, a solution, a spray, a wax-based stick, or a towelette. In addition, the compositions contemplated by this invention can include one or more compatible cosmetically acceptable adjuvants commonly used and known by the skilled practitioner, such as colorants, fragrances, emollients, humectants, preservatives, vitamins, chelators, thickeners, perilla oil or perilla seed oil (WO 01/66067 to a “Method of Treating a Skin Condition,” incorporated herewith in its entirety) and the like, as well as other botanicals such as aloe, chamomile, and the like, and as further described below.

Also, embraced by the invention are transdermal modes of delivery, such as patches and the like, with or without suitable penetration enhancers. The methods and compositions embodied by the invention provide a means by which the adipose septa protein modulators can be effectively administered in a transdermal system. Accordingly, a transdermal means of delivering a composition or formulation (often with a penetration enhancing composition) to the skin is that of the transdermal patch or a similar device as known and described in the art. Transdermal patches are designed to deliver an effective amount of compound across a user's skin. Transdermal patches typically involve a liquid, gel, solid matrix, or pressure-sensitive adhesive carrier into which one or more of the above noted extracts may be incorporated. Patch formulations and preparations are well known in the art. See for example “Dermatological and Transdermal Formulations” (Drugs and the Pharmaceutical Sciences, Vol 119) by Kenneth A Walters (Editor), Marcel Dekker and “Transdermal Drug Delivery” (Drugs & the Pharmaceutical Sciences) by Richard H. Guy (Editor), Jonathan Hadgraft (Editor) 2nd Rev& ex edition Marcel Dekker and “Mechanisms of Transdermal Drug Delivery” (Drugs & the Pharmaceutical Sciences, Vol 83) edited by Russell O. Potts and Richard H. Guy (1997) all of which are incorporated herein by reference in their entirety. Examples of such devices are disclosed in U.S. Pat. Nos. 5,146,846; 5,223,262; 4,820,724; 4,379,454; and 4,956,171; and U.S. Patent Publication No. US20110300198, all of which are incorporated herein by reference in their entirety. The transdermal mode of storing and delivering the compositions onto the skin, including hair, and forming the active composition is convenient and well-suited for the purposes of an embodiment of the present invention. In another method, the application is through a sustained release vehicle, carrier, or diluent, e.g., a topically applied sustained released patch. When a topical patch is used, the patch may be applied to the desired area for extended period of time. The extended period of time may be greater than one hour, and in certain embodiments the extended period of time is overnight, i.e., when the user is sleeping. In a further embodiment of the current invention, the transdermal patch may be applied to skin exhibiting cellulite or at risk for exhibiting cellulite, i.e., the buttocks, thighs, hips, or limbs for extended periods of time, at least one day, two or more days, at least a week, or longer if necessary in order to provide prolonged exposure to the adipose septa protein modulators in order to achieve the desired enhancements in the strength or elasticity of the adipose septa.

The compositions can include a cosmetically acceptable vehicle. Such vehicles may take the form of any known in the art suitable for application to skin and may include water (e.g., deionized water); vegetable oils; mineral oils; esters such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, in one embodiment organomodified derivatives; hydrocarbon oils such as mineral oil, petrolatum, isoeicosane and polyisobutene; polyols such as propylene glycol, glycerin, butylene glycol, pentylene glycol and hexylene glycol; waxes such as beeswax and botanical waxes; or any combinations or mixtures of the foregoing.

The vehicle may comprise an aqueous phase, an oil phase, an alcohol, a silicone phase or mixtures thereof. The cosmetically acceptable vehicle may also comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. The emulsion may include an emulsifier, such as a nonionic, anionic or amphoteric surfactant.

The oil phase of the emulsion in one embodiment has one or more organic compounds, including emollients; humectants (such as butylene glycol, propylene glycol, Methyl gluceth-20, and glycerin); other water-dispersible or water-soluble components including thickeners such as veegum or hydroxyalkyl cellulose; gelling agents, such as high MW polyacrylic acid, i.e. CARBOPOL 934; and mixtures thereof. The emulsion may have one or more emulsifiers capable of emulsifying the various components present in the composition.

The compounds suitable for use in the oil phase include without limitation, vegetable oils; esters such as octyl palmitate, isopropyl myristate and isopropyl palmitate; ethers such as dicapryl ether; fatty alcohols such as cetyl alcohol, stearyl alcohol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; natural or synthetic waxes; and the like. Suitable hydrophobic hydrocarbon oils may be saturated or unsaturated, have an aliphatic character and be straight or branched chained or contain alicyclic or aromatic rings. The oil-containing phase may be composed of a singular oil or mixtures of different oils.

Hydrocarbon oils include those having 6-20 carbon atoms may be utilized, in one embodiment having 10-16 carbon atoms. Representative hydrocarbons include decane, dodecane, tetradecane, tridecane, and C₈₋₂₀ isoparaffins. Paraffinic hydrocarbons are available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the tradename Permethyl 99A™ are also contemplated to be suitable. Various commercially available C₁₆ isoparaffins, such as isohexadecane (having the tradename Permethyl®) are also suitable. Examples of volatile hydrocarbons include polydecanes such as isododecane and isodecane, including for example, Permethyl-99A (Presperse Inc.) and the C₇-C₈ through C₁₂-C₁₅ isoparaffins such as the Isopar Series available from Exxon Chemicals. A representative hydrocarbon solvent is isododecane.

The oil phase may comprise one or more waxes, including for example, rice bran wax, carnauba wax, ouricurry wax, candelilla wax, montan waxes, sugar cane waxes, ozokerite, polyethylene waxes, F ischer-Tropsch waxes, beeswax, microcrystalline wax, silicone waxes, fluorinated waxes, and any combination thereof.

Non-limiting emulsifiers include emulsifying waxes, emulsifying polyhydric alcohols, polyether polyols, polyethers, mono- or di-ester of polyols, ethylene glycol mono-stearates, glycerin mono-stearates, glycerin di-stearates, silicone-containing emulsifiers, soya sterols, fatty alcohols such as cetyl alcohol, acrylates, fatty acids such as stearic acid, fatty acid salts, and mixtures thereof. Emulsifiers may include soya sterol, cetyl alcohol, stearic acid, emulsifying wax, acrylates, silicone containing emulsifiers and mixtures thereof. Other specific emulsifiers that can be used in the composition of the present invention include, but are not limited to, one or more of the following: C₁₀₋₃₀ alkyl acrylate crosspolymer; Dimethicone PEG-7 isostearate, acrylamide copolymer; mineral oil; sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the INCI Ingredient Dictionary and Handbook 11^{th} Edition 2006, the disclosure of which is hereby incorporated by reference in its entirety.

These emulsifiers typically will be present in the composition in an amount from about 0.001% to about 10% by weight, in particular in an amount from about 0.01% to about 5% by weight, and in one embodiment, from about 0.1% to about 3% by weight.

The oil phase may comprise one or more volatile and/or non-volatile silicone oils. Volatile silicones include cyclic and linear volatile dimethylsiloxane silicones. In one embodiment, the volatile silicones may include cyclodimethicones, including tetramer (D₄), pentamer (D₅), and hexamer (D₆) cyclomethicones, or mixtures thereof. Particular mention may be made of the volatile cyclomethicone-hexamethyl cyclotrisiloxane, octamethyl-cyclotetrasiloxane, and decamethyl-cyclopentasiloxane. Suitable dimethicones are available from Dow Corning under the name Dow Corning 200® Fluid and have viscosities ranging from 0.65 to 600,000 centistokes or higher. Suitable non-polar, volatile liquid silicone oils are disclosed in U.S. Pat. No. 4,781,917, herein incorporated by reference in its entirety. Additional volatile silicones materials are described in Todd et al., “Volatile Silicone Fluids for Cosmetics”, Cosmetics and Toiletries, 91:27-32 (1976), herein incorporated by reference in its entirety. Linear volatile silicones generally have a viscosity of less than about 5 centistokes at 25° C., whereas the cyclic silicones have viscosities of less than about 10 centistokes at 25° C. Examples of volatile silicones of varying viscosities include Dow Corning 200, Dow Corning 244, Dow Corning 245, Dow Corning 344, and Dow Corning 345, (Dow Corning Corp.); SF-1204 and SF-1202 Silicone Fluids (G.E. Silicones), GE 7207 and 7158 (General Electric Co.); and SWS-03314 (SWS Silicones Corp.). Linear, volatile silicones include low molecular weight polydimethylsiloxane compounds such as hexamethyldisiloxane, octamethyltrisiloxane, decamethyltetrasiloxane, and dodecamethylpentasiloxane, to name a few.

Non-volatile silicone oils will typically comprise polyalkylsiloxanes, polyarylsiloxanes, polyalkylarylsiloxanes, or mixtures thereof. Polydimethylsiloxanes are non-volatile silicone oils. The non-volatile silicone oils will typically have a viscosity from about 10 to about 60,000 centistokes at 25° C., in one embodiment between about 10 and about 10,000 centistokes, and in one embodiment still between about 10 and about 500 centistokes; and a boiling point greater than 250° C. at atmospheric pressure. Non limiting examples include dimethyl polysiloxane (dimethicone), phenyl trimethicone, and diphenyldimethicone. The volatile and non-volatile silicone oils may optionally be substituted with various functional groups such as alkyl, aryl, amine groups, vinyl, hydroxyl, haloalkyl groups, alkylaryl groups, and acrylate groups, to name a few.

The water-in-silicone emulsion may be emulsified with a nonionic surfactant (emulsifier) such as, for example, polydiorganosiloxane-polyoxyalkylene block copolymers, including those described in U.S. Pat. No. 4,122,029, the disclosure of which is hereby incorporated by reference in its entirety. These emulsifiers generally comprise a polydiorganosiloxane backbone, typically polydimethylsiloxane, having side chains comprising -(EO)ₘ— and/or —(PO)ₙ— groups, where EO is ethyleneoxy and PO is 1,2-propyleneoxy, the side chains being typically capped or terminated with hydrogen or lower alkyl groups (e.g., C₁₋₆, typically C₁₋₃). Other suitable water-in-silicone emulsifiers are disclosed in U.S. Pat. No. 6,685,952, the disclosure of which is hereby incorporated by reference herein. Commercially available water-in-silicone emulsifiers include those available from Dow Corning under the trade designations 3225C and 5225C FORMULATION AID; SILICONE SF-1528 available from General Electric; ABIL EM 90 and EM 97, available from Goldschmidt Chemical Corporation (Hopewell, Va.); and the SILWET series of emulsifiers sold by OSI Specialties (Danbury, Conn.).

Examples of water-in-silicone emulsifiers include, but are not limited to, dimethicone PEG 10/15 crosspolymer, dimethicone copolyol, cetyl dimethicone copolyol, PEG-15 lauryl dimethicone crosspolymer, laurylmethicone crosspolymer, cyclomethicone and dimethicone copolyol, dimethicone copolyol (and) caprylic/capric triglycerides, polyglyceryl-4 isostearate (and) cetyl dimethicone copolyol (and) hexyl laurate, and dimethicone copolyol (and) cyclopentasiloxane. In one embodiment examples of water-in-silicone emulsifiers include, without limitation, PEG/PPG-18/18 dimethicone (trade name 5225C, Dow Corning), PEG/PPG-19/19 dimethicone (trade name BY25-337, Dow Corning), Cetyl PEG/PPG-10/1 dimethicone (trade name Abil EM-90, Goldschmidt Chemical Corporation), PEG-12 dimethicone (trade name SF 1288, General Electric), lauryl PEG/PPG-18/18 methicone (trade name 5200 FORMULATION AID, Dow Corning), PEG-12 dimethicone crosspolymer (trade name 9010 and 9011 silicone elastomer blend, Dow Corning), PEG-10 dimethicone crosspolymer (trade name KSG-20, Shin-Etsu), dimethicone PEG-10/15 crosspolymer (trade name KSG-210, Shin-Etsu), and dimethicone PEG-7 isostearate.

The water-in-silicone emulsifiers typically will be present in the composition in an amount from about 0.001% to about 10% by weight, in another embodiment in an amount from about 0.01% to about 5% by weight, and in a further embodiment in an amount below 1% by weight.

The aqueous phase of the emulsion may include one or more additional solvents, including lower alcohols, such as ethanol, isopropanol, and the like. The volatile solvent may also be a cosmetically acceptable ester such as butyl acetate or ethyl acetate; ketones such as acetone or ethyl methyl ketone; or the like.

The oil-containing phase will typically comprise from about 10% to about 99%, about 20% to about 85%, or from about 30% to about 70% by weight, based on the total weight of the emulsion, and the aqueous phase will typically comprise from about 1% to about 90%, about 5% to about 70%, or from about 20% to about 60% by weight of the total emulsion.

The compositions may include liposomes. The liposomes may comprise other additives or substances and/or may be modified to more specifically reach or remain at a site following administration.

The composition may optionally comprise other cosmetic actives and excipients, obvious to those skilled in the art including, but not limited to, fillers, emulsifying agents, antioxidants, surfactants, film formers, chelating agents, gelling agents, thickeners, emollients, humectants, moisturizers, vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants, depigmenting agents, hypopigmenting agents, preservatives (e.g., DMDM Hydantoin/Iodopropynylbutylcarbonate), stabilizers, pharmaceutical agents, photostabilizing agents, neutralizers (e.g., triethanolamine) and mixtures thereof. In addition to the foregoing, the cosmetic compositions of the invention may contain any other compound for the treatment of skin disorders.

Colorants may include, for example, organic and inorganic pigments and pearlescent agents. Suitable inorganic pigments include, but are not limited to, titanium oxide, zirconium oxide and cerium oxide, as well as zinc oxide, iron oxide, chromium oxide and ferric blue. Suitable organic pigments include barium, strontium, calcium, and aluminium lakes and carbon black. Suitable pearlescent agents include mica coated with titanium oxide, with iron oxide, or with natural pigment.

Various fillers and additional components may be added. Fillers are normally present in an amount of about 0 weight % to about 20 weight %, based on the total weight of the composition, in one embodiment about 0.1 weight % to about 10 weight %. Suitable fillers include without limitation silica, treated silica, talc, zinc stearate, mica, kaolin, Nylon powders such as Orgasol™, polyethylene powder, Teflon™, starch, boron nitride, copolymer microspheres such as Expancel™ (Nobel Industries), Polytrap™ (Dow Corning) and silicone resin microbeads (Tospearl™ from Toshiba), and the like.

In one embodiment of the invention, the compositions may include additional skin actives such as, but not limited to, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, salicylic acid or salicylates, thiodipropionic acid or esters thereof, and advanced glycation end-product (AGE) inhibitors.

In a specific embodiment, the composition may comprise at least one additional botanical, such as, for example, a botanical extract, an essential oil, or the plant itself. Suitable botanicals include, without limitation, extracts from Abies pindrow, Acacia catechu, Anogeissus latifolia, Asmunda japonica, Azadirachta indica, Butea frondosa, Butea monosperma, Cedrus deodara, Emblica officinalis, Ficus benghalensis, Glycyrrhiza glabra, Ilex purpurea Hassk, Inula racemosa, Ligusticum chuangxiong, Ligusticum lucidum, Mallotus philippinensis, Mimusops elengi, Morinda citrifolia, Moringa oleifera, Naringi crenulata, Nerium indicum, Psoralea corylifolia, Stenoloma chusana, Terminalia bellerica, tomato glycolipid and mixtures thereof.

The composition may comprise additional active ingredients having anti-aging benefits, as it is contemplated that synergistic improvements may be obtained with such combinations. Exemplary anti-aging components include, without limitation, botanicals (e.g., Butea frondosa extract); thiodipropionic acid (TDPA) and esters thereof; retinoids (e.g., all-trans retinoic acid, 9-cis retinoic acid, phytanic acid and others); hydroxy acids (including alpha-hydroxyacids and beta-hydroxyacids), salicylic acid and salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); barrier function enhancing agents (e.g., ceramides, glycerides, cholesterol and its esters, alpha-hydroxy and omega-hydroxy fatty acids and esters thereof, etc.); collagenase inhibitors; and elastase inhibitors; to name a few.

Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof.

In another embodiment, the topical compositions of the present invention may also include one or more of the following: a skin penetration enhancer, an emollient, a skin plumper, an optical diffuser, a sunscreen, an exfoliating agent, and an antioxidant.

An emollient provides the functional benefits of enhancing skin smoothness and reducing the appearance of fine lines and coarse wrinkles. Examples include isopropyl myristate, petrolatum, isopropyl lanolate, silicones (e.g., methicone, dimethicone), oils, mineral oils, fatty acid esters, cetyl ethylhexanoate, C12-15 alkyl benzoate, isopropyl isostearate, diisopropyl dimer dillinoeate, or any mixtures thereof. The emollient may be present from about 0.1 wt % to about 50 wt % of the total weight of the composition.

A skin plumper serves as a collagen enhancer to the skin. An example of a suitable, and in one embodiment, skin plumper is palmitoyl oligopeptide. Other skin plumpers are collagen and/or other glycosaminoglycan (GAG) enhancing agents. When present, the skin plumper may comprise from about 0.1 wt % to about 20 wt % of the total weight of the composition.

An optical diffuser is a particle that changes the surface optometrics of skin, resulting in a visual blurring and softening of, for example, lines and wrinkles. Examples of optical diffusers that can be used in the present invention include, but are not limited to, boron nitride, mica, nylon, polymethylmethacrylate (PMMA), polyurethane powder, sericite, silica, silicone powder, talc, Teflon, titanium dioxide, zinc oxide, or any mixtures thereof. When present, the optical diffuser may be present from about 0.01 wt % to about 20 wt % of the total weight of the composition.

A sunscreen for protecting the skin from damaging ultraviolet rays may also be included. In one embodiment sunscreens are those with a broad range of UVB and UVA protection, such as octocrylene, avobenzone (Parsol 1789), octyl methoxycinnamate, octyl salicylate, oxybenzone, homosylate, benzophenone, camphor derivatives, zinc oxide, and titanium dioxide. When present, the sunscreen may comprise from about 0.01 wt % to about 70 wt % of the composition.

Suitable exfoliating agents include, for example, alpha-hydroxyacids, beta-hydroxyacids, oxaacids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. In one embodiment exfoliating agent is glycolic acid. When present, the exfoliating agent may comprise from about 0.1 wt % to about 80 wt % of the composition.

Antioxidants scavenge free radicals from skin, protecting the skin from environmental aggressors. Examples of antioxidants that may be used in the present compositions include compounds having phenolic hydroxy functions, such as ascorbic acid and its derivatives/esters; alpha-hydroxyacids; beta-carotene; catechins; curcumin; ferulic acid derivatives (e.g. ethyl ferulate, sodium ferulate); gallic acid derivatives (e.g., propyl gallate); lycopene; reductic acid; rosmarinic acid; tannic acid; tetrahydrocurcumin; tocopherol and its derivatives (e.g., tocopheryl acetate); uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present invention may comprise an antioxidant in one embodiment from about 0.001 wt % to about 10 wt %, and in one embodiment from about 0.01 wt % to about 5 wt %, of the total weight of the composition.

Other conventional additives include: vitamins, such as tocopherol and ascorbic acid; vitamin derivatives such as ascorbyl monopalmitate; thickeners such as hydroxyalkyl cellulose; gelling agents; structuring agents such as bentonite, smectite, magnesium aluminum silicate and lithium magnesium silicate; metal chelating agents such as EDTA; pigments such as zinc oxide and titanium dioxide; colorants; emollients; and humectants.

In one embodiment, the composition of the invention comprising an adipose septa protein modulator may have a pH between about 1 and about 8. In certain embodiments, the pH of the composition will be acidic, i.e., less than 7.0, and in other embodiments will be between about 2 and about 7, in further embodiments will be between about 3.5 and about 5.5.

The invention provides a method for treating skin irregularities of aging due to weakening of adipose septa and/or excessive or abnormal growth of adipose septa through the topical application of a composition comprising an adipose septa protein modulator, in one embodiment in a cosmetically acceptable vehicle, over the affected area for a period of time sufficient to reduce, ameliorate, reverse or prevent the skin irregularity. This method is particularly useful for treating cellulite, sagging facial or neck skin, in particular periorbital bulging, and scleroderma. In certain embodiments, the adipose septa protein modulators are used in a non-therapeutic manner.

Cosmetic compositions taught herein can be applied to an area of skin affected by cellulite to improve the appearance of the skin. An improvement may involve a reduction in appearance of lumpiness and/or unevenness, characteristic of cellulite, in one embodiment reducing what is known as the “cottage cheese” or “orange peel” look. Further, areas of cellulite tend to bulge, pit, and dimple when squeezed or compressed, as occurs when legs are crossed when seated, which can worsen the appearance of cellulite areas. In some embodiments, an improvement involves a reduction in this pitting appearance of cellulite upon squeezing, so that the look of cellulite on the legs appears reduced even when sitting with the legs crossed. An improvement may also involve reducing the visible depth and/or intensity of cellulite. In certain embodiments, known evaluative scales can be used to determine the initial severity of the cellulite and gauge the improvement after treatment with the cosmetic compound of the current invention. One such scale is the Nurnberger-Muller scale, which provides a scale of: Stage 0—no dimpling when the subject is standing and lying down—a pinch test can show folds in the skin, but no cellulite is visible; Stage 1—no dimpling when the subject is standing or lying down however when pinched, the skin shows signs of cellulite; Stage 2—dimpling is present when standing only; and Stage 3—dimpling is visible when both standing and lying down; cellulite may be painful depending on where it is located and how much fat is present. In such instances an improvement in appearance may be viewed as a reduction in stage based on the scale, i.e. going from about stage 2 to about stage 1.

Cellulite tends to accumulate on certain body regions, e.g., on the thighs and buttocks of many women, as well as on the abdomen, hip and/or upper arm region. In some embodiments, the extent of the area affected by cellulite is reduced, such that smaller areas of the thigh, buttocks, abdomen, hip, and/or upper arm region remain visibly affected. In certain embodiments, one or more of such regions becomes free of visible signs of cellulite following treatment with a composition described herein. In some particular embodiments, the composition is not applied to skin affected by acne or inflammation.

In some embodiments, a method is provided for reducing the re-occurrence of cellulite in an area that was previously affected by cellulite, but showing little or no signs of cellulite currently. Reducing the re-occurrence refers to delaying the recurrence of any cellulite on a previously-affected area, or reducing the extent of cellulite that re-appears on the area, such that any recurrent cellulite is less visible than previous amounts.

Similarly, compositions comprised of the adipose septa protein modulators of the current invention may be applied to sagging areas of the facial or neck skin such as around the eyes, cheeks, jowls, forehead, and neck in order to obtain an improvement in the appearance of the skin, i.e., a reduction in the severity of sagging, a reduction in the puffiness of the sagging, a reduction in the size of the area affected by sagging. Further, the compositions of the current invention may be applied to the area of skin at risk of sagging in an effort to prevent, forestall, or delay the appearance of such sags. In one embodiment, the method of the current invention is used to address periorbital bulging, i.e. a sagging below and/or above the eye.

In some embodiments, the cosmetic compositions for combating signs of unwanted subcutaneous fat can further comprise additional anti-lipid agents. For example, the cosmetic composition comprising a Carnitine Palmitoyl Transferase-1 (CPT-1) stimulator (e.g. the leaf extract of Averrhoa carambola) in an amount effective (or amounts effective) to improve the appearance of skin may further comprise at least one other anti-lipid agent, including one other anti-cellulite agent. It is contemplated that synergistic improvements may be obtained with such combinations, in some embodiments.

Exemplary anti-cellulite agents include, without limitation, phosphodiesterase inhibitors, such as xanthine analogs, caffeine, aminophylline, and theophylline; adenylate cyclase activators, such as forskolin and Coleus forskohlii extract; lipolysis stimulators, such as hawthorne extract and cola extract; beta adrenergic receptor agonists, such as isoproterenol; alpha-2-adrenergic antagonists, such as yohimbine and Ginkgo biloba extract; perilla oil (see, e.g., U.S. Pat. No. 7,410,658, incorporated herein by reference in its entirety); carnitine and/or creatine (see, e.g., US 2007/0264205 entitled “Cosmetic Composition having Carnitine Creatinate and Methods for Using,” incorporated herein by reference in its entirety). In some embodiments, additional actives may include a collagen stimulator and/or an elastin stimulator, e.g., a substance that stimulates elastin production, and/or a glycosaminoglycan enhancer. Examples of collagen, elastin and glycosaminoglycan enhancers include, e.g., fennel extract, carrot extract, and alfalfa extract. In some embodiments, the additional actives may include a collagenase inhibitor and/or elastase inhibitor. In some embodiments, the invention relates to synergistic action of one or more compositions described herein with perilla oil, e.g., to provide enhanced anti-cellulite benefits to skin.

In some embodiments, the cosmetic compositions can further comprise at least one collagen and/or elastin stimulator. Such collagen or elastin stimulators are effective in, for example, providing improvement in procollagen and/or collagen production and/or improvement in maintenance and remodeling of elastin.

Improvements in the dermal architecture, e.g., by strengthening adipose septa, reduces the likelihood of fat nodules “blebbing” between connective tissue fibers or septa, which is believed to lead to the characteristic unsightly appearance of cellulite. Further, lower levels of subcutaneous fat further reduce the likelihood of such blebbing. As cellulite is believed to result from a combination of enlarged fat tissue and weak dermal structure, combating cellulite through these multiple approaches (the adipose septa proteins of the current invention in combination with the above-noted anti-lipid and anti-cellulite agents), as described herein, can provide superior results compared with products that utilize only one approach.

Accordingly, the invention provides novel mechanisms of action to improve the appearance of cellulite, and thus provides for potent anti-cellulite compositions for use thereon.

Without wishing to be bound by any particular theory, it is believed that the compositions of the present invention enhance and improve the aesthetic appearance of skin by improving/strengthening the adipose septa within the subcutaneous adipose tissue thereby reducing the presence of adipose bulges underlying the skin.

In a further embodiment of the current invention, the adipose septa protein modulators disclosed herein may be used to downregulate the production or over production of one or more adipose septa proteins that may lead to undesirable effects on the skin. See e.g. Small leucine-rich proteoglycans, decorin and fibromodulin, are reduced in postburn hypertrophic scar, Wound Repair and Regeneration, May 17, 2011. The over production of the adipose septa proteins may lead to excessive growth or abnormal growth of the septa resulting in undesirable effects such as keratosis, skin diseases or disorders characterized by overgrowths of the septa, scleroderma, and/or wrinkles. Examples of such conditions may include, scars such as keloid scars and cutaneous effects of scleroderma such as sclerodactylia, acrosclerosis, and/or telangiectasis.

In one embodiment of the current invention, the modulator is applied to skin experiencing over production of an adipose septa protein such as skin afflicted by scleroderma. The modulator may be applied topically to obtain relief from pitted scarring, thickening and hardening of the skin, swelling of the skin such as sclerodactylia or acrosclerosis, shiny appearance of the skin, tightening of the skin across the underlying bone, telangiectasis, and/or darkening of the skin associated with scleroderma.

Further, the modulators of the current invention may impart anti-aging benefits as well by treating, ameliorating or preventing the weakening and/or compromised adipose septa associated with aging. The signs of aging are manifested in the wrinkles, folds, and pouches such as those found along the forehead; cheeks; around the eyes, and most prominently under the eyes; at the corners of the mouth; under the chin; or on the neck. As noted above, these undesirable features are due in part to the weakening of the septa and the underlying fat pads succumbing to the force of gravity. The method of the current invention may be used to rejuvenate, repair, and/or strengthen the adipose septa or fibrous septa and thereby prevent, forestall, ameliorate, and/or treat these undesirable signs of aging.

The composition will typically be applied to the skin one, two, or three times daily for as long as is necessary to achieve desired smoothing results. The treatment regimen may comprise daily application for at least one week, at least two weeks, at least four weeks, at least eight weeks, or at least twelve weeks. Chronic treatment regimens are also contemplated.

In a specific embodiment, the adipose septa protein modulator is provided in a pharmaceutically, physiologically, cosmetically, and dermatologically-acceptable vehicle, diluent, or carrier, where the composition is topically applied to an affected area of skin and left to remain on the affected area in an amount effective for improving the condition and aesthetic appearance of skin.

The method of the invention may be employed prophylactically to forestall aging including in patients that have not manifested signs of skin aging, most commonly individuals under 25 years of age. The method may also reverse or treat signs of aging once manifested as is common in patients over 25 years of age.

### EXAMPLES

### Example 1

### Proteomic Analysis of Adipose Septa

Abdominal subcutaneous adipose tissue of female Caucasians was used to prepare adipose septa. Adipose septa were carefully isolated from adipose to avoid the associated fat and blood vessel and washed several times with ice-cold phosphate buffered saline to remove blood. Adipose septa were ground and denatured in 8M urea solution, reduced and alkylated with 0.25% triethylphosphine and 1% iodoethanol in 50% acetonitrile. The denatured and reduced septa were digested in 10 mM ammonium bicarbonate buffer containing trypsin (Promega, WI) at 37° C. overnight. The tryptic peptides were injected onto the C18 column (Xbridge C18 2.5 μM-2.1 mm×5 cm) using Surveyor HPLC pump. Peptides were eluted with a linear gradient from 3 to 40% acetonitrile (in water) developed over 120 min at 50° C., at flow rate of 200 μl/min, and effluent was electro-sprayed into the LTQ mass spectrometer (Thermo-Fisher, MA). Database search was performed against the IPI human database (V3.60) using Sequest and X!Tandem algorithms and identification confidence was calculated by a published method (Higgs, R. E. et al. (2007) J. Proteome Res. 4, 1758-1767). The Peptide ID Confidence that assigned a protein into a “high” or “moderate” classification was used to identify septa proteins. Proteins with best peptide having a confidence between 90-100% are assigned to the “high” category. Proteins with best peptide having a confidence between 75-89% are assigned to the “moderate” category. All of the septa proteins identified from adipose belong to the “high” category. Moreover, the gene expression of each septa protein was confirmed by qPCR in primary human subcutaneous adipocytes. The resulting “high” proteins identified are listed in Table 1 below.

TABLE 1
Adipose Septa Proteins

| Septa Proteins | Gene |
|---|---|
| Biglycan | BGN |
| Mimecan | OGN |
| Prolargin | PRELP |
| Asporin | ASPN |
| Dermatopontin | DPT |
| Nidogen 1 and 2 | NID1, NID2 |
| Galectin-1 | LGALS1 |
| MAGP-4 | MFAP4 |
| Fibromodulin | FMOD |
| Laminin Beta 2 | LAMB2 |
| Decorin | DCN |
| Lumican | LUM |
| Fibronectin | FN1 |

### Example 2

### Assay for Gene Expression of Septa Protein

For each of the prospective adipose septa protein modulators noted below, primary human preadipocytes were plated confluent in preadipocyte medium. Next day differentiation was initiated by adding adipocyte differentiation medium and cells were allowed to differentiate for 7 days. On the day 7 of differentiation, 1 volume of differentiation medium was left and 3 volumes of adipocyte maintenance medium were added. Cells were incubated for 7 days (14 days of differentiation total) and treated with given concentration of active on day 8, 10, and 13 of differentiation. Day 10 and 13 treatment was added in adipocyte maintenance medium. After treatment, cells were washed with ice cold PBS, collected into RLT lysis buffer and RNA was extracted using RNeasy Mini Kit (Qiagen, Valencia, Calif.) following manufactures recommendations. 200 ng of RNA was used for cDNA synthesis using High Capacity cDNA Reverse Transcription Kit (Life Technologies, Carlsbad, Calif.). 1 μL of undiluted cDNA was used per qPCR reaction. Primers used for detection of septa protein mRNA were purchased from Life technologies (Carlsbad, Calif.) and are listed below in Table 2.

TABLE 2
Septa Protein Primers

| | | Primer Life Technology |
|---|---|---|
| Septa Proteins | Gene | Catalog Number |
| Biglycan | BGN | Hs00959143_m1 |
| Mimecan | OGN | Hs00925258_m1 |
| Prolargin | PRELP | Hs01941580_s1 |
| Asporin | ASPN | Hs01550903_m1 |
| Dermatopontin | DPT | Hs00355056_m1 |
| Nidogen 1 and 2 | NID1, NID2 | Hs00201233_m1 (NID1) |
| | | Hs00159600_m1 (NID2) |
| Galectin-1 | LGALS1 | Hs00355202_m1 |
| MAGP-4 | MFAP4 | Hs00412974_m1 |
| Fibromodulin | FMOD | Hs00157619_m1 |
| Laminin Beta 2 | LAMB2 | Hs00158642_m1 |
| Decorin | DCN | Hs00754870_s1 |
| Lumican | LUM | Hs00929860_m1 |
| Fibronectin | FN1 | Hs00287359_m1 |

Expression of septa protein mRNA was normalized to 18S rRNA. The conditions of q-PCR are an incubation step at 50° C. for 2 minutes and an enzyme activation step at 95° C. for 10 minutes; followed by 45 cycles of 95° C. for 30 seconds and 60° C. for 1 minute. CT value is obtained from the software of the Stratagene MX2005P.

TABLE 3
Adipose Septa Protein Modulators

| | | Biglycan | | Mimecan | | Prolargin | | Dermatopontin | |
|---|---|---|---|---|---|---|---|---|---|
| | Conc. | % change | p | % change | p | % change | p | % change | p |
| Septa Protein Modulator | (%) | vs. control | value | vs. control | value | vs. control | value | vs. control | value |
| Amorphophallus campanulatus | 0.01 | 54.78 | 0.0001 | 105.12 | 0.05 | — | — | — | — |
| Medemia nobilis | 0.001 | 52.15 | 0.00 | 163.19 | 0.02 | 87.325 | 0.0023 | −45.70 | 0.0825 |
| Erythrina indica | 0.1 | −68.994 | 0.0001 | 78.37 | 0.01 | −59.54 | 0.0000 | −40.96 | 0.0353 |
| Ixora chinensis | 0.1 | −82.912 | 0.0001 | 229.45 | 0.04 | 87.338 | 0.0000 | 53.90 | 0.0986 |
| Operculina turpethum | 0.1 | −84.481 | 0.00002 | 55.87 | 0.24 | 64.571 | 0.0001 | 47.93 | 0.1954 |
| Portulaca oleracea | 0.1 | −77.438 | 0.00001 | 153.15 | 0.0034 | −59.509 | 0.1 | 7.60 | 0.0002 |
| Tiliacora triandra | 0.1 | −79.8718 | 0.00004 | 51.83 | 0.01 | −25.1 | 0.0007 | 2.18 | 0.7305 |
| Palmitoyl Lysyl Aminovaleroyl Lysine | 0.01 | 22.14 | 0.00 | 76.81 | 0.03 | 8.579 | 0.2636 | −13.82 | 0.4445 |
| Melicope | 0.005 | 67.41 | 0.00 | 94.95 | 0.0031 | −74.827 | 4.3E−08 | −41.62 | 0.0176 |
| Butea frondosa | 0.01 | 150.01 | 0.00 | 300.06 | 0.00 | — | — | — | — |

As shown in the chart above, each of the prospective adipose septa protein modulators were found to modulate the activity of the septa proteins biglycan, mimecan, prolargin, and dermatopontin. In particular, biglycan was upregulated by greater than 50% by Amorphophallus campanulatus, Melicope, and Medemia nobilis; and by greater than 100% by Butea frondosa. However, Erythrinia indica, Ixora chinensis, Operculina turpethum, Portulaca oleracea, and Tiliacora triandra all led to a greater than 50% downregulation of biglycan. Mimecan was upregulated by each of the adipose septa modulators: greater than 50% by Erythrinia indica, Operculina turpethum, Triliacora triandra, palmitoyl lysylaminovaleroyl lysine, and Melicope, and by greater than 100% by Amorphophallus campanulatus, Ixora chinensis, Portulaca oleracea, and Butea frondosa. Medemia noblis, Ixora chinensis, and Operculina turpethum upregulated prolargin by greater than 50%, whereas Eryhtrinia indicia, Portulaca oleracea, and Melicope downregulated prolargin by greater than 50%. Dermantopotin was upregulated by Operculina turpethum, Portulaca oleracea, and Triliacora triandra, and by greater than 50% by Ixora chinensis. The remaining adipose septa protein modulators assayed downregulated dermantopotin.

### Example 3

### Exemplary HPLC Protocol

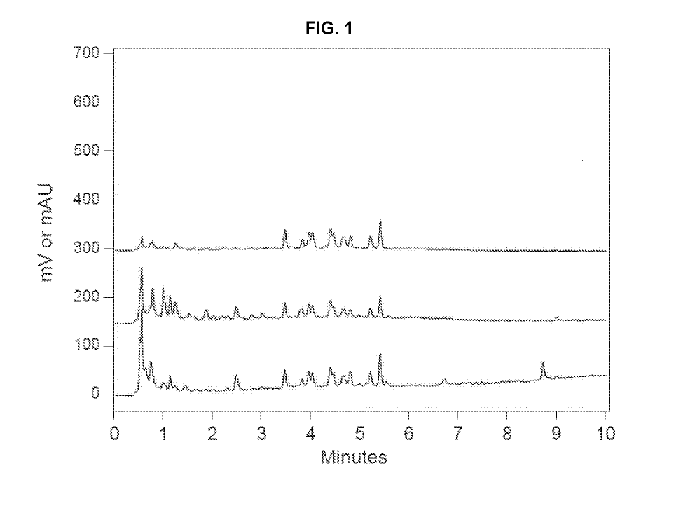

Extracts were generally characterized by high performance liquid chromatography. A sample size of approximately 5 mg/mL was dispersed in 25/75 MeOH/H₂O and sonicated. The characterization was performed on a Zorbax SBC-18 column (7.5 cm×4.6 mm, 3.5 um particle size) and detection was achieved using diode array UV absorbance, 260 nm 300 nm and 360 nm, with lines on FIG. 1 depicted in ascending order and 260 nm on bottom. Operating conditions were flow rate 1.5 ml/min; temperature, 40° C.; sample injection volume, 20 μL, and time of run, 19 minutes. The mobile phase gradient used was as follows. In one embodiment, the extracted composition of a compound, in substantial isolation, exhibits an HPLC profile substantially similar to that depicted herein.

TABLE 4
Mobile Phase Gradient

| Time | Phase |
|---|---|
| 0 Minutes: | 15% Methanol(Solvent B)/85% Water with 1% acetic |
| | acid (Solvent A) |
| 10 Minutes: | 95% Methanol/5% Water with 1% Acetic acid. |
| 15 Minutes: | 15% Methanol/85% Water with1% Acetic acid. |
| 15.01 Minutes | 95% Methanol/5% Water with1% Acetic acid. |
| 19 Minutes: | 15% Methanol/85% Water with1% Acetic acid |

### Example 4

### Preparation of Extracts

A. Preparation of Melicope Extract

The process generally follows a combination of steam distillation and hydro-distillation, as it uses partial immersion of the biomass and boiling water steam, although it is typically referred to as steam distillation. Steam distillation relies on heat to open the oil glands in the plant and the essential oil and water to mix. Then the increase in vapor pressure and corresponding reduction in boiling point which occurs with the mixing of 2 immiscible liquids allows the essential oil, which would normally have a boiling point of greater than 200° C., to boil at less than 100° C.

Leaves and terminal branchlets were cut from stands of Melicope growing wild on the north coast of New South Wales (NSW), Australia. Approximately 2.102 Kg of leaf biomass was loaded loosely into a 20 Liter reaction vessel set up as a distillation unit with receiver condenser and 500 ml separating flask. 4 Liters of Hot Water were added to the vessel and additional heat added via a hot plate. The flow rate of the condenser water was adjusted to give a distillate temperature of at least 50° C. in the separating funnel. The essential oil floated on the water. At approximately 1 hour intervals the water was drained off and returned to the vessel. The distillation was stopped after 8.5 hours when no further oil was distilled. The water in the separating flask was drained and the essential oil tapped off. The 2.102 Kg of leaf biomass produced 3.35 g of essential oil, a yield of 0.16%. The essential oil was found to have a relative density at 20° C. of 0.950 and a Refractive Index of 1.510 at 20° C.

As noted in the remaining specification, modifications and adaptations of this extraction process are possible, particularly during a scale-up to larger volumes for production.

The essential oil content of the Melicope extracts were identified using the procedures detailed within Brophy, J. Essent. Oil Res. (2004), vol. 16, pp 286-293, hereby incorporated by reference in its entirety.

TABLE 5
Essential Oil Profiles for Melicope Extracts

| Melicope | | Bicyclo- | | |
|---|---|---|---|---|
| Batch | Caryophyllene | germacrene | Zierone | Evodone |
| Extract 1 | 5.7 | 0.9 | 28 | 14.1 |
| Extract 2 | 5.7 | 0.9 | 26.8 | 14.5 |
| Extract 3 | 4.4 | 0.9 | 27.2 | 12.8 |
| Extract 4 | 4.5 | 0.9 | 29.1 | 11.8 |
| Extract 5 | 4.9 | 1 | 27.8 | 13.5 |
| Extract 6 | 5.3 | 0.9 | 27.4 | 13.3 |

### B. Preparation of Tiliacora Triandra Extract.

Tiliacora triandra may be extracted from natural raw materials using methods of aqueous organic solvent extraction as is well known in the art. Two such extraction processes are set forth below.

### 1. Extraction of Tiliacora Triandra by Ethanol

An extract was obtained by extracting the vine of the Tiliacora triandra plant using an ethanol extraction scheme. Briefly, the vines of Tiliacora triandra Diels were first manually ground into small particles resulting in a powder of about 250 grams per flask (2 flasks). The ground powder was then extracted with 80% ethanol (2×2,000 ml per flask). After filtering and vacuum evaporation, the total concentrated extract was lyophilized resulting in an ethanolic extract of 50 grams. Tannins were removed resulting in an ethanolic extract of Tiliacora triandra of 46.04 grams.

### 2. Extraction of Tiliacora Triandra by Hexane

An extract was obtained by extracting the vine of the Tiliacora triandra plant using a hexane extraction scheme. Briefly, the vines of the Tiliacora triandra were first manually ground into small particles resulting in a powder of about 250 grams per flask (2 flasks). The ground powder was then extracted with 100% hexane (2×2,000 ml per flask). After filtering and vacuum evaporation, the total concentrated extract was dried by hot air oven at 40° C. resulting in an hexanolic extract of Tiliacora triandra of 0.61 grams.

A HPLC trace of a representative Tiliacora triandra extract is found at FIG. 1.

C. Preparation of Medemia Nobilis Extract.

Preparation of Medemia nobilis extract is generally described in U.S. patent application Ser. Nos. 13/305,779 and 13/216,626, filed on Dec. 30, 2010 and Aug. 24, 2011, respectively. Medemia nobilis leaves and stems are extracted with water/ethanol, and filtered to generate a Medemia nobilis raw extract. The extract is then concentrated to aqueous suspension, which is let stand overnight at 4 C. The concentrated aqueous suspension is then precipitated and filtered with solid fraction removed, yielding a filtered aqueous filtered solution. Butanol is then added to the filtered aqueous suspension, then a liquid/liquid extraction is performed with subsequent removal of the organic phase. The remaining aqueous phases is then concentrated, dried, and irradiated to yield a dried purified Medemia nobilis extract, which may then be resuspended for further use (in one embodiment, as an aqueous resuspension).

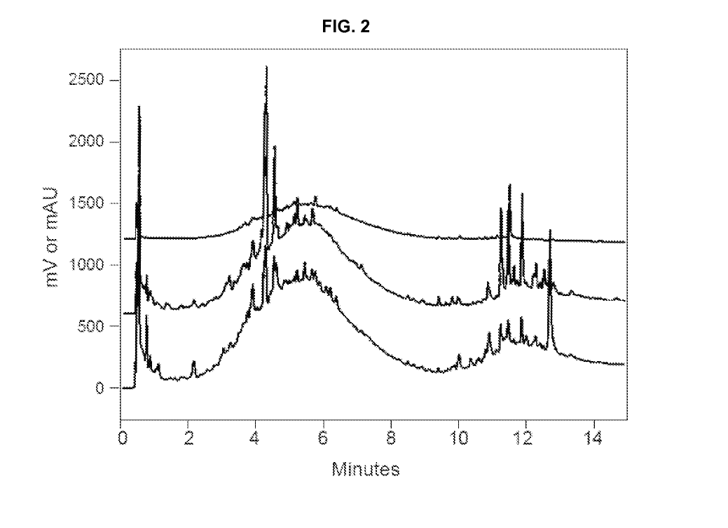

A HPLC trace of a representative Medemia nobilis extract is found at FIG. 2.

D. Preparation of Ixora Chinensis Extract

Preparation of Ixora chinensis extract is generally described in U.S. patent application Ser. No. 13/158,947, filed on Jun. 30, 2010, the entirety of which is incorporated by reference for all purposes and U.S. patent application Ser. No. 13/324,150, filed on Dec. 13, 2011, the entirety of which is incorporated by reference for all purposes. An extract is obtained by extracting the dry chopped plant of Ixora chinensis Lamk. using an ethanol extraction followed by a further extraction with hexane. Briefly, the chopped plant of Ixora chinensis is first manually ground into small particles resulting in a powder of about 250 grams. The ground powder is then extracted with 50% ethanol. After filtering and vacuum evaporation, the total concentrated extract is diluted with water, centrifuged and filtered. The liquid is then thrice extracted with hexane, the hexane upper layer being discarded and the aqueous layer being lyophilized resulting in an extract of about 90 grams.

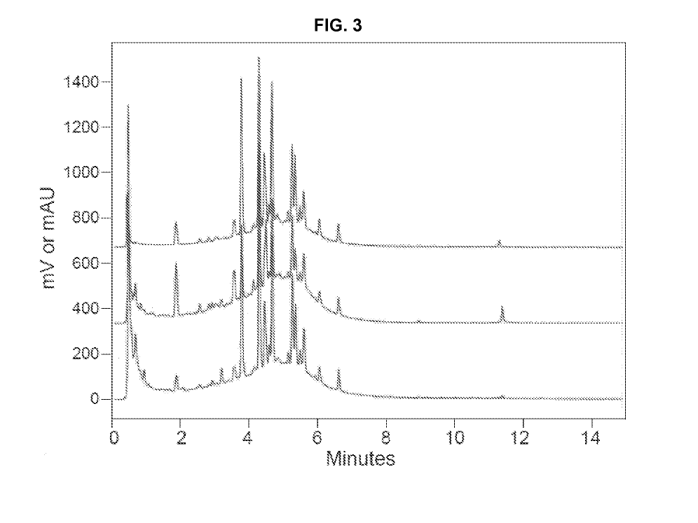

A HPLC trace of a representative Ixora chinensis extract is found at FIG. 3.

E. Preparation of Operculina Turpethum Extract

Preparation of Operculina turpethum extract is generally described in U.S. patent application Ser. No. 13/158,947, filed on Jun. 30, 2010. Operculina turpethum leaves and stems are extracted with water/ethanol, and filtered to generate a Operculina turpethum raw extract. The extract is then concentrated to aqueous suspension, which is let stand overnight at 4 C. The concentrated aqueous suspension is then precipitated and filtered with solid fraction removed, yielding a filtered aqueous filtered solution. Butanol is then added to the filtered aqueous suspension, then a liquid/liquid extraction is performed with subsequent removal of the organic phase. The remaining aqueous phases is then concentrated, dried, and irradiated to yield a dried purified Operculina turpethum extract, which may then be resuspended for further use (in one embodiment, as an aqueous resuspension).

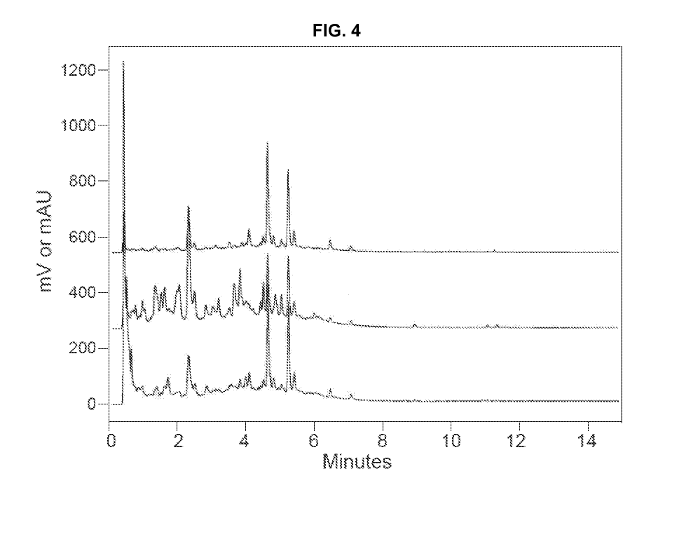

A HPLC trace of a representative Operculina turpethum extract is found at FIG. 4.

F. Preparation of Erythrina Indicia Extract

Preparation of Erythrina indicia extract is generally described in U.S. patent application Ser. No. 12/648,581, filed on Dec. 29, 2009; Ser. No. 12/966,098, filed on Dec. 29, 2009; and Ser. No. 12/827,001, filed on Jun. 30, 2010. Erythrina indicia leaves and stems are extracted with water/ethanol, and filtered to generate a Erythrina indicia raw extract. The extract is then concentrated to aqueous suspension, which is let stand overnight at 4 C. The concentrated aqueous suspension is then precipitated and filtered with solid fraction removed, yielding a filtered aqueous filtered solution. Butanol is then added to the filtered aqueous suspension, then a liquid/liquid extraction is performed with subsequent removal of the organic phase. The remaining aqueous phases are then concentrated, dried, and irradiated to yield a dried purified Erythrina indicia extract, which may then be resuspended for further use (in one embodiment, as an aqueous resuspension).

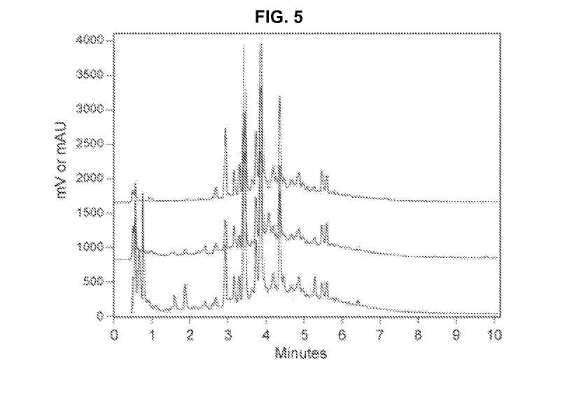

A HPLC trace of a representative Erythrina indicia extract is found at FIG. 5.

G. Preparation of Portulaca Oleracea Extract

Preparation of Portulaca oleracea extract is generally described in U.S. patent application Ser. No. 12/827,001, filed on Jun. 30, 2010. Portulaca oleracea leaves and stems are extracted with water/ethanol, and filtered to generate a Portulaca oleracea raw extract. The extract is then concentrated to aqueous suspension, which is let stand overnight at 4 C. The concentrated aqueous suspension is then precipitated and filtered with solid fraction removed, yielding a filtered aqueous filtered solution. Butanol is then added to the filtered aqueous suspension, then a liquid/liquid extraction is performed with subsequent removal of the organic phase. The remaining aqueous phases is then concentrated, dried, and irradiated to yield a dried purified Portulaca oleracea extract, which may then be resuspended for further use (in one embodiment, as an aqueous resuspension).

H. Preparation of Amorphophallus Campunulatus Extract.

Preparation of Amorphophallus campunulatus extract is generally described in U.S. patent application Ser. No. 13/158,947, filed on Jun. 30, 2010. Amorphophallus campunulatus leaves and stems are extracted with water/ethanol, and filtered to generate a Amorphophallus campunulatus raw extract. The extract is then concentrated to aqueous suspension, which is let stand overnight at 4 C. The concentrated aqueous suspension is then precipitated and filtered with solid fraction removed, yielding a filtered aqueous filtered solution. Butanol is then added to the filtered aqueous suspension, then a liquid/liquid extraction is performed with subsequent removal of the organic phase. The remaining aqueous phases is then concentrated, dried, and irradiated to yield a dried purified Amorphophallus campunulatus extract, which may then be resuspended for further use (in one embodiment, as an aqueous resuspension).

Exemplary Compositions

A. Exemplary Anti-Cellulite Compositions

Cosmetic compositions comprising an adipose septa protein modulator for topical application to skin exhibiting or at risk of exhibiting cellulite are provided in Table 6.

TABLE 6
Sample Anti-Cellulite Cosmetic Composition

Ingredient
Aesthetic modifier
Emollient
Emulsifier
Anti-inflammation agent
Chelater
Coolant
Elastin stimulator
Exfoliator
Fragrance
Humectant
Microcirculation enhancer
Neutralizer
Preservative
Sunscreen
Collagenase/elastinase inhibitor
Hawthorne (Crataeg. monog.) Fruit. Extract
Coffee Seed Extract
Soybean (Glycine soja) Extract
Celosia cristata Extract & Prunella vulgaris Extract
L-Carnitine Hydrochloride
Averrhoa carambola Leaf Extract
Septa protein modulator
Demineralized water

B. Exemplary Anti-Aging Facial Cosmetic Composition

Cosmetic compositions comprising an adipose septa protein modulator for topical application to areas of the face exhibiting or at risk of exhibiting signs of aging due to a reduction in the quality of the adipose septa are provided in Table 7.

TABLE 7
Sample Anti-aging Facial Cosmetic Composition

Ingredient
Aesthetic modifier
Emollient
Emulsifier
Anti-inflammation agent
Chelater
Coolant
Elastin stimulator
Exfoliator
Fragrance
Humectant
Microcirculation enhancer
Neutralizer
Preservative
Sunscreen
Collagenase/elastinase inhibitor
Phytol
Antioxidant
Fennel Extract
Carrot extract
Pomegranate extract
Thiodipropionic acid (TDPA)
Green tea polyphenol
L-4 Thiazolylanine
Septa protein modulator
Demineralized water

These compositions are believed to be effective to treat, reverse, ameliorate and/or prevent signs of skin aging, specifically, the compositions are believed to reduce the appearance of skin irregularities, such as cellulite on the body and under-eye bags on the face. The compositions of Table 4 are applied to skin in need of treatment, by which is meant skin in need of an anti-cellulite benefit, and in particular skin exhibiting irregularities due to a weakening of the adipose septa such as cellulite on the abdomen, thighs, buttocks or limbs. The compositions of Table 5 are applied to the facial skin in need of treatment, by which is meant skin in need of an anti-aging benefits, and in particular skin exhibiting irregularities due to a weakening of the adipose septa such as periorbital bulging. These cosmetic compositions may be applied directly to the affected areas of skin, i.e., the cellulite or periorbital bulging.

The cosmetic compositions are applied to the skin, cellulite and/or eye bag one, two or three times daily for as long as is necessary to achieve desired results, which treatment regimen may comprise daily application for at least one week, at least two weeks, at least four weeks, at least eight weeks, or at least twelve weeks. Alternatively, the exemplary cosmetic compositions may be used in chronic treatment of the skin, cellulite and/or eye bags in need thereof.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We hereby claim:

1. A method for improving the appearance of skin affected by a skin irregularity resulting from changes in adipose septa comprising topically applying thereto an effective amount of at least one modulator of an adipose septa protein, in a cosmetically acceptable vehicle for a time sufficient to achieve an improvement in the appearance of said skin.

2. The method according to claim 1, wherein the modulator upregulates an adipose septa protein.

3. The method according to claim 1, wherein the modulator downregulates an adipose septa protein.

4. The method according to claim 1, wherein said skin irregularity is cellulite sagging facial skin, sagging neck skin, or scleroderma.

5. The method according to claim 4, wherein said skin irregularity is cellulite.

6. The method according to claim 5, wherein said achievement in the appearance of the skin is selected from the group consisting of:
(a) reduction in the appearance of cellulite lumpiness and/or unevenness;
(b) reduction in pitting appearance of cellulite upon squeezing;
(c) reduction in the extent of area affected by cellulite;
(d) prevention or delay in the recurrence of cellulite;
(e) improvement in collagen disposition; or
(f) improvement in adipocyte/fat tissue disposition;

7. The method of claim 4, wherein said skin irregularity is sagging of the facial skin.

8. The method according to claim 8, wherein said sagging of the facial skin is periorbital bulging.

9. The method of claim 7, wherein said achievement in the appearance of the skin is selected from the group consisting of:
(a) improvement in adipocyte/fat tissue disposition;
(b) reduction in sagging of the facial skin; or
(c) reduction in the extent of the area affected by sagging.

10. The method of claim 1, wherein the modulator comprises palmitoyl lysyl aminovaleroyl lysine, an extract of Amorphophallus campanulantus, an extract of Mademia nobilis, an extract of Erythrina indica, an extract of Ixora chinensis, an extract of Operculina turpethum, an extract of Portulaca oleracea, an extract of Tiliacora triandra, an extract of Melicope, an extract of Butea frodosa, or combination thereof.

11. The method of claim 10, wherein the modulator is not an extract of Tiliacora triandra or Melicope.

12. The method of claim 1, wherein the adipose septa protein comprises asporin, biglycan, decorin, dermatopontin, fibromodulin, fibronectin, galectin-1, laminin beta 2, lumican, MAGP-4, mimecan (osteoglycin), nidogen-1, nidogen-2, or prolargin.

13. The method of claim 1, wherein the modulator is in combination with at least one other anti-lipid agent.

14. The method according to claim 13, wherein said at least one other anti-lipid agent comprises a carnitine palmitoyl transferase-1 stimulator.

15. The method of claim 1, wherein the modulator is in combination with at least one anti-cellulite agent.

16. The method of claim 15, wherein the at least one anti-cellulite agent is selected from the group consisting of a phophodiesterase inhibitor, an adenylate cyclase activator, a lipolysis stimulator, a beta-adrenergic receptor agonist, an alpha-2-adreneric receptor antagonist, perilla oil, carnitine, creatine, or combination thereof.

17. The method according to claim 1, wherein said modulator is in combination with at least one collagen and/or elastin stimulator.

18. The method according to claim 1, wherein said modulator is in combination with a retinoid.

19. The method of claim 1, wherein an effective amount of the modulator is about 0.001% to about 25% by weight.

20. A method for screening active agents useful for improving the aesthetic appearance of skin comprising assaying candidate substances for ability to modify adipose septa protein expression.

21. A method of treating the skin comprising topically applying to an area of the skin in need thereof an effective amount of an active agent that modulates adipose septa protein expression, wherein the ability of said active agent to modulate adipose septa protein expression has been determined by an assay which measures the level of mRNA encoding adipose septa protein in a cell that has been contacted with said active agent.
